(19) United States
(12) Patent Application Publication
(10) Pub. No.: US 2023/0046622 A1
(43) Pub. Date: Feb. 16, 2023

(54) Integrated High Flow Oxygen Concentration Management System

(71) Applicant: Telesair, Inc., Irvine, CA (US)

(72) Inventor: Qing Wang, Irvine, CA (US)

(21) Appl. No.: 17/397,949

(22) Filed: Aug. 9, 2021

(51) Int. Cl. C01B 13/02; A61M 16/10; A61M 16/16

(52) U.S. Cl. CPC C01B 13/027; A61M 16/101; A61M 16/16; C01B 2210/0018; C01B 2210/0046; A61M 2202/0208; A61M 2016/1025

(57) ABSTRACT

An oxygen production unit is provided. The oxygen production unit, in various embodiments, includes a separation unit configured to separate oxygen from nitrogen in received gaseous particles; a control unit configured to control an amount of the separated oxygen to be released to a user of the oxygen production unit, and a nitrogen release unit configured to facilitate release of the separated nitrogen within oxygen production unit and/or into an environment where the oxygen production unit is located. The oxygen production unit in those embodiments can automatically determine an amount of oxygen to be produced and/or delivered to a user; a control and facilitate release of nitrogen into the environment to enhance safety; to facilitate swapping of nitrogen piece(s) in the oxygen production unit to prolong a lifetime of the oxygen production unit, and/or achieve any other benefits.

### FIELD OF THE INVENTION

The invention generally relates to oxygen production techniques for home and/or non-professional use.

### BACKGROUND OF THE INVENTION

An oxygen generator is a device that separates oxygen from compressed air using special selective adsorptive technology called pressure swing adsorption (PSA). The compressed air used in the oxygen generation process has a similar composition to ambient environmental air with 21% oxygen and 78% nitrogen. The oxygen contained in the compressed air is allowed to flow through a zeolite molecular sieve which retains nitrogen resulting in high purity oxygen at gas production outlets of the oxygen generator.

The terms oxygen generator and oxygen concentrator are quite often used interchangeably and essentially mean the same thing. Generically speaking, an oxygen concentrator is a term used to define a smaller scale oxygen generation device (portable home concentrators) while an oxygen generator is a term more commonly used to describe equipment that processes large quantities of oxygen used in industrial manufacturing.

### SUMMARY OF THE INVENTION

In various embodiments, an oxygen production unit is provided. The oxygen production unit, in those embodiments, includes a separation unit configured to separate oxygen from nitrogen in received gaseous particles; a control unit configured to control an amount of the separated oxygen to be released to a user of the oxygen production unit, and a nitrogen release unit configured to facilitate release of the separated nitrogen into an environment where the oxygen production unit is located. The oxygen production unit in those embodiments can automatically determine an amount of oxygen to be produced and/or delivered to a user; a control and facilitate release of nitrogen into the environment to enhance safety; to facilitate swapping of nitrogen piece(s) in the oxygen production unit to prolong a lifetime of the oxygen production unit, and/or achieve any other benefits.

In some embodiments, the nitrogen release unit includes a first outlet connectable to a first inlet of a cannula to form a first channel, and a second outlet connectable to a second inlet of the cannula to form a second channel. In those embodiments, the first channel facilitates the separated nitrogen into the ambient environment through the cannula, and the second channel facilitates the separated oxygen into a nose of the user through the cannula, the first channel and the second channel are insulated from each other.

In some embodiments, the nitrogen release unit includes an outlet openable to the ambient environment, and is configured to facilitate the release of the separated nitrogen into the environment directly. In those embodiments, the nitrogen release unit includes a container configured house a piece absorbed with the separated nitrogen. In those embodiments, facilitating the release of the nitrogen includes: generating a pressure difference in the container to facilitate the separated nitrogen to be released into the environment. In some embodiments, the nitrogen release unit includes an outlet openable to the ambient environment. In those embodiments, the separated nitrogen is released from the container to the ambient environment through the outlet.

In some embodiments, the control unit is further configured to determine a nitrogen level in the piece exceeds a threshold level and generate a signal to alert that nitrogen level in the piece has exceeded the threshold level. In those embodiments, the control unit is configured to: receive a value indicating pre-determined amount of oxygen to be released to the user; receive a value indicating a physiological parameter regarding the user; and determine the amount of the oxygen to be released to the user based on the pre-determined amount of oxygen and the physiological parameter.

In some embodiments, determining the amount of the oxygen to be released to the user based on the pre-determined amount of oxygen and the physiological parameter includes: determining an adjustment value in the amount of the oxygen to be released to the user based on the pre-determined amount of oxygen and the physiological parameter to obtain.

In some embodiments, the oxygen production unit includes a mix unit configured to: receive a value indicating the amount of the oxygen to be release to the user from the control unit; obtain an amount of gas based on the value; and mix the separated oxygen with the amount of gas to achieve the amount of the oxygen to be release to the user.

In some embodiments, the oxygen production unit includes a humidification unit configured to humidify the separated oxygen before being released to the user. In some embodiments, the oxygen production unit includes a heat unit configured to heat the separated oxygen before being released to the user. In some embodiments, the control unit is configured to control the amount of the separated oxygen to be released to the user based on the physiological parameters. In those embodiments, the control unit is configured to control the amount of the separated oxygen to be released to the user based on the upper physiological limit and the lower physiological limit. The physiological parameters includes blood oxygen saturation, respiratory rate, heart rate, and/or blood pressure.

Other objects and advantages of the invention will be apparent to those skilled in the art based on the following drawings and detailed description.

### BRIEF DESCRIPTION OF THE DRAWINGS

FIG. 1A generally illustrates an overview of an oxygen production unit in accordance with the disclosure

FIGS. 1B-C illustrates examples of the oxygen production unit shown in FIG. 1A.

FIG. 2 illustrates an embodiment of a cannula shown in FIG. 1B.

FIG. 3 illustrates another embodiment of the cannula shown in FIG. 1B.

FIG. 4 illustrates an embodiment of the nitrogen release unit shown in FIG. 1B.

FIG. 5 illustrates another embodiment of the nitrogen release unit shown in FIG. 1B.

FIG. 6 illustrates still another embodiment of the oxygen production unit shown in FIG. 1B.

FIG. 7 illustrates yet another embodiment of the oxygen production unit shown in FIG. 1B.

FIG. 8 illustrates an embodiment of oxygen production unit shown in FIG. 1C.

FIG. 9 illustrates another embodiment of the oxygen production unit shown in FIG. 1B.

FIG. 10 illustrates still another embodiment of the oxygen production unit shown in FIG. 1C.

FIG. 11 illustrates yet another embodiment of the oxygen production unit shown in FIG. 1B.

FIG. 12 illustrates another embodiment of the oxygen production unit shown in FIG. 1A.

FIG. 13 illustrates still another embodiment of the oxygen production unit shown in FIG. 1A.

FIG. 14 illustrates yet another embodiment of the oxygen production unit shown in FIG. 1A.

FIG. 15 illustrates one example method for producing oxygen for a user in accordance with the disclosure

### DETAILED DESCRIPTION

The following disclosure provides many different embodiments, or examples, for implementing different features of the provided subject matter. Specific examples of components and arrangements are described below to simplify the present disclosure. These are, of course, merely examples and are not intended to be limiting. For example, the formation of a first feature over or on a second feature in the description that follows may include embodiments in which the first and second features are formed in direct contact, and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed. For a particular repeated reference numeral, cross-reference may be made for its structure and/or function described and illustrated herein.

Further, spatially relative terms, such as “beneath,” “below,” “lower,” “above,” “upper” and the like, may be used herein for ease of description to describe one element or feature's relationship to another element(s) or feature(s) as illustrated in the figures. The spatially relative terms are intended to encompass different orientations of the device in use or operation in addition to the orientation depicted in the figures. The apparatus may be otherwise oriented (rotated 90 degrees or at other orientations) and the spatially relative descriptors used herein may likewise be interpreted accordingly.

Oxygen generation system may be designed to concentrate oxygen from a gas supply such as ambient air by selectively removing nitrogen to supply an oxygen-enriched product gas stream. In medical applications, the concentrated oxygen produced by the oxygen concentration management system can be delivered to patients for treatment of breathing-related disorders such as asthma, pneumonia, respiratory distress syndrome, bronchopulmonary dysplasia, chronic obstructive pulmonary disease. Oxygen delivery for treatment of breathing-related disorders and diseases can be managed by a respiratory therapist trained in critical care and cardio-pulmonary medicine.

As oxygen generation systems have become more wide-spread, determining an adequate amount of separated oxygen delivered to the patients has garnered some attention. The challenge is to determine an adequate amount of separated oxygen produced by the oxygen concentration management system in order to deliver to patients with breathing-related disorders. With the delivered amount of separated oxygen, oxygen concentration in the blood of the patients can be adjusted to normal level. If the amount of separated oxygen delivered to the patient is too high, the patient may experience oxygen toxicity, resulting in lung damage, trouble breathing, or even death in severe cases. If the amount of separated oxygen delivered to the patient is too low, on the other hand, the patient may not receive enough amount of separated oxygen for the treatment, resulting in deterioration of the breathing-related diseases. While determining an adequate amount of separated oxygen can be managed by a respiratory therapist, use of oxygen production unit in a non-professional setting may be limited due to shortage and cost of respiratory therapists and other medical resources available in a non-professional use setting (such as at a patient's home). Traditionally, on-demand respiration therapists are needed for non-professional settings such that the therapists would carry specialized equipment to the non-professional setting, and provide respiration services to the patients in the non-professional setting. One motivation behind the present disclosure is to enable a user or patient to use oxygen production in a non-professional setting where a respiration therapist may not be available onsite to provide his/her service. In accordance with the disclosure, various embodiments provide an oxygen production unit that is configured to adjust oxygen production amount automatically and/or remotely by a respiration therapist. Such an oxygen production unit may bring convenience and reduce costs for a user or patient to use the oxygen production unit in the non-professional setting.

For achieving this, one motivation behind the present disclosure is to automatically determine an adequate amount of separated oxygen to facilitate h non-professional use of an oxygen production unit when a respiration therapist is not available control the oxygen production unit. In some embodiments, a closed-loop feedback control circuit is deployed in such a unit to control the amount of separated oxygen to be released to the user or patient based on an amount of oxygen predetermined for the user or patient. In some embodiments, control mechanisms are made available for a respiration therapist to be able to control the oxygen production unit remotely.

In various embodiments, pressure swing adsorption technology is used in the oxygen production unit in accordance with the disclosure to separate oxygen from ambient air under pressure according to molecular characteristics and affinity for an adsorbent material. In various embodiments, adsorbent materials such as zeolites are used as a trap to adsorb nitrogen and produce high density oxygen_{[SF1]}. Although pressure swing adsorption technology can carry out oxygen separation on a small scale, its application in a non-professional setting is not without challenges. For example, its application is limited to processes that require lower rate oxygen. As another example, zeolites adsorbents are inefficient and unable to produce high oxygen flow rate with pressure swing adsorption technology. Thus, various embodiments provide improvements to the pressure swing adsorption technology to provide higher oxygen flow rate to address such constraints.

Besides the ability to automatically produce and adjust an adequate amount of separated oxygen, other considerations for non-professional use may include safety consideration and life-time of the oxygen production unit. Non-professional use of an oxygen generation system may cause safety concerns as the environment of non-professional use may not possess professional safety measures as in hospital-like environments. For example, a user in the environment of home-based oxygen generation system may produce smoke when smoking cigarette, which may cause fire accidents when the oxygen concentration generation system is in use.

Thus, another motivation behind the present disclosure is to enhance safety when an oxygen production unit is used in home or in a non-professional setting. In various embodiments, this is achieved by releasing nitrogen from oxygen production unit into specific locations in the ambient environment to reduce high oxygen density in the ambient air, and prevent or reduce risks of fire hazards which may be caused by the high oxygen density in the ambient air. One motivation behind the present disclosure is to have the oxygen production unit in accordance with the disclosure designed structurally to release nitrogen to the ambient environment at those location.

As mentioned above, non-professional use an oxygen production system could cause shortened life-time of such a system due to lack of professional care. One care item for an oxygen production system is that a user should know when to swap MOF (Metal Organic Frameworks) pieces when they are saturated with nitrogen above a threshold density. In various embodiments, the oxygen production unit in accordance with the disclosure is configured to facilitate swapping of nitrogen piece(s) in a nitrogen release unit of the oxygen production unit as the piece(s) becomes saturated with nitrogen. This can prolong a lifetime of the oxygen production unit configured to release nitrogen into the environment. In one embodiment, two MOF pieces are used in the oxygen production unit in accordance with the disclosure. One piece is used to provide oxygen to the patient by absorbing nitrogen and allowing oxygen to pass through; when the amount of nitrogen in this piece is increased to saturation, a second piece is swapped in to continue providing oxygen to the patient. In that embodiment, the one with saturated nitrogen is placed in a container with lower pressure to release nitrogen into the container. The air in the container with higher percentage of nitrogen component is released through an air pipe to patient's head area to reduce oxygen level in the head area to reduce the fire and other risk.

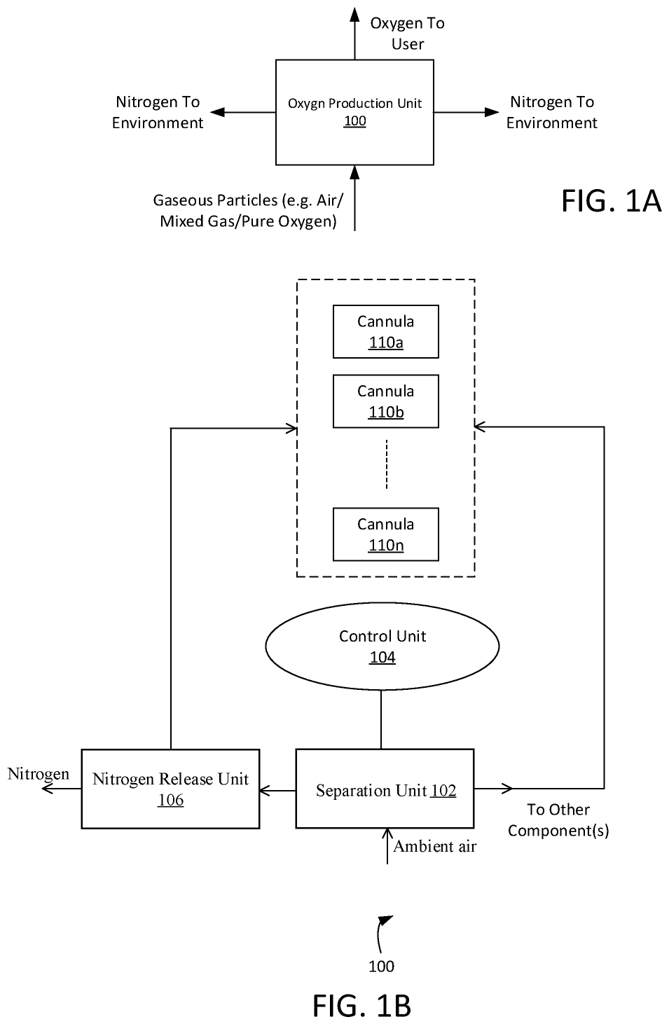

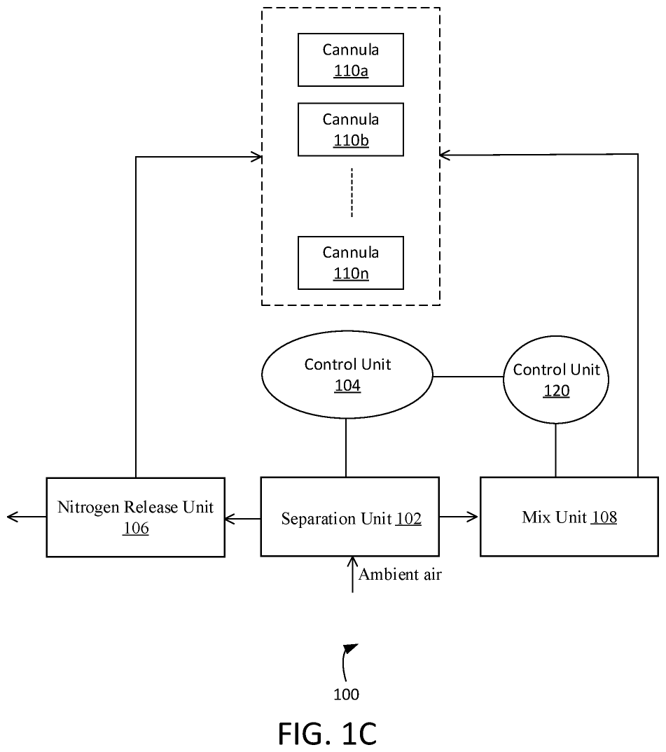

FIG. 1A generally illustrates an overview of an oxygen production unit 100 in accordance with the disclosure. As mentioned, the oxygen production unit 100 may be used in a non-professional setting by a user for providing oxygen of higher purify than that in the ambient air to the user. The oxygen production unit 100 in accordance with the disclosure is configured to receive air (such as ambient air), gas (such mixed gas), pure oxygen, and/or any other gaseous particles containing oxygen. The oxygen production unit 100 in accordance with the disclosure is configured to produce oxygen with desired purity by separating oxygen from the gaseous particles received. The oxygen production unit 100 in accordance with the disclosure is configured to release nitrogen to an environment where the oxygen production unit 100 is located, for example around the user and/or around the oxygen production unit 100. The nitrogen released by the oxygen production unit 100 may reduce oxygen density in the environment. For example, the nitrogen released by the oxygen production unit 100 can create a shield around the oxygen production unit 100 to protect the oxygen production unit 100 from fire hazards or any other hazards. As another example, the nitrogen released by the oxygen production unit 100 can reduce oxygen concentration around the user of oxygen production unit 100 as the user breathe out concentrated oxygen during the use of oxygen production unit 100.

### Example System

With an overview of the oxygen production unit 100 in accordance the disclosure having been generally described and illustrated, attention is now directed to FIG. 1B, which illustrates an example the oxygen production unit 100 shown in FIG. 1A. As shown, in this example the oxygen production unit 100 includes a separation unit 102, a control unit 104, a nitrogen release unit 106, one or more cannulas such as 110a, b and n shown, and/or any other components. The separation unit 102 is configured to receive and/or obtain ambient air from ambient environment of the oxygen production unit 100 through one or more separation unit inlets. Upon receiving and/or obtaining the ambient air from the ambient environment of the oxygen production unit 100, the separation unit 102 is configured to use adsorption materials to attract nitrogen molecules onto the surfaces of the adsorption materials more strongly than oxygen molecules, resulting in simultaneous separation of nitrogen and oxygen from the ambient air. The adsorption materials may include metal-organic frameworks, covalent organic frameworks, and/or any other adsorption materials.

In implementation, the separation unit 102 may be configured to release separated oxygen one or more other components of the oxygen production unit 100. For example, in some embodiments, the oxygen production unit 100 can include a mix unit (not shown in FIG. 1) configured to blend the oxygen produced by the oxygen production unit 100 with one or more other gaseous particles to produce desired mixed gases to be provided to the user. For instance, in one embodiment, the oxygen produced by the oxygen production unit 100 is released to the mix unit to be blended with ambient air such that a desired concentration of oxygen can be achieved for delivery to the user.

In this example, the separation unit 102 may be configured to receive and/or obtain separation unit control signals from the control unit 104. The separation unit control signals may include power on and off signal, ambient air reception on and off signal, runtime control signal, emergent stop control signal, and/or any other separation unit control signals. As mentioned, in various implementation, the separation unit 102 incorporates MOF (Metal Organic Frameworks) or a COF (Covalent Organic Frameworks) structure, whereas injected air pass through the separation unit 102.

In some embodiments, the separation unit 102 comprises a first separation unit outlet connectable to a first nitrogen release unit inlet of the nitrogen release unit 106 to form a first separation unit to nitrogen release unit channel, and a second separation unit outlet connectable to a first mix unit inlet of the mix unit 108 to form a first separation unit to mix unit channel. The first separation unit to nitrogen release unit channel may be configured to facilitate separated nitrogen from the separation unit 102 into the nitrogen release unit 106. The first separation unit to mix unit channel may be configured to facilitate separated oxygen from the separation unit 102 to the mix unit 108.

The control unit 104 is configured to control one or more aspects of oxygen production by the separation unit 102. For example, as mentioned above, the control unit 104 can be configured to control power on and off of the separation unit 102. As another example, the control unit 104 may be configured to determine an amount of separated oxygen to be release from the oxygen production unit 100. Still as another example, the control unit 104 may be configured to control a period of time the separation unit 102 engages in oxygen separation operation to facilitate releasing of oxygen from the oxygen production unit 100. Yet till as another example, the control unit 104 may be configured to monitor a level of nitrogen saturation

In various embodiments, the oxygen production unit 100 in accordance with the present disclosure may include a mix unit 108. FIG. 1C shows one example of oxygen production unit 100 that includes a mix unit 108. An amount of separated oxygen to be released to user of the oxygen production unit 100 may be referred to a ratio between volume of oxygen and volume of all air constituents in air to be released to user of the oxygen production unit 100. In some embodiments, the mix unit 108 may be configured to receive and/or obtain the amount of separated oxygen from the control unit 104 in order to release oxygen corresponding to the received and/or obtained amount of separated oxygen. In this example, the amount of separated oxygen may be determined based on one or more physiological parameters of the user of the oxygen production unit 100. The physiological parameters may include blood oxygen saturation, respiratory rate, heart rate, blood pressure, and/or any other physiological parameters. In various implementation, as shown in FIG. 1C, an additional control unit 106 can be included in the oxygen production unit 100 to control the mxi unit 108. For example, the control unit 120 can be configured to control one or more aspects of the mix unit 108. For example, the control unit 106 can be configured to control an amount of ambient air to be mixed with the oxygen released from the separation unit 102 such that an optimal ratio of oxygen is released to the patient.

In various implementations, the control unit 104 and/or control unit 120 may have dedicated circuitry/logic for efficiently processing machine-readable commands/instructions. In some embodiments, the control unit 104 and/or control unit 120 unit may include a micro-processor configured to execute the machine-readable commands/instructions. In some embodiments, the control unit 104 and/or control unit 120 may include storage such as a memory storage configured to store one or more machine-readable instructions. Various operations described and illustrated herein as being attributed to the control unit 104 are stored in such a storage as commands/instructions. For example, a manufacturer of the oxygen production unit 100 may store those commands/instructions in advance during a manufacturing stage of the oxygen production unit 100.

The nitrogen release unit 106 may be configured facilitate release of the separated nitrogen, for example, into ambient air of the oxygen production unit 100, and/or within the oxygen production unit 100. The nitrogen release unit 106 may be configured to receive and/or obtain nitrogen release unit control signals from the control unit 104 and/or control unit 120. The nitrogen release unit control signals may include power on and off signal, nitrogen reception on and off signal, runtime control signal, emergent stop control signal, air pressure adjustment control signal, and/or any other nitrogen release unit control signals. In this example, the nitrogen release unit 106 may be configured to release the separated nitrogen from the oxygen production unit 100 to the ambient air in order to reduce high oxygen density in the ambient air and prevent fire incidents which may be caused by the high oxygen density. However, this is not the only case. In some examples, the separated nitrogen is released within the oxygen production unit 100 to create an inertia environment. This cam help improve operation safety of the oxygen production unit 100.

The control unit 104 and/or control unit 120, in various embodiments, are configured to control the nitrogen release unit 106, the separation unit 102, the mix unit 108, and/or any other components.

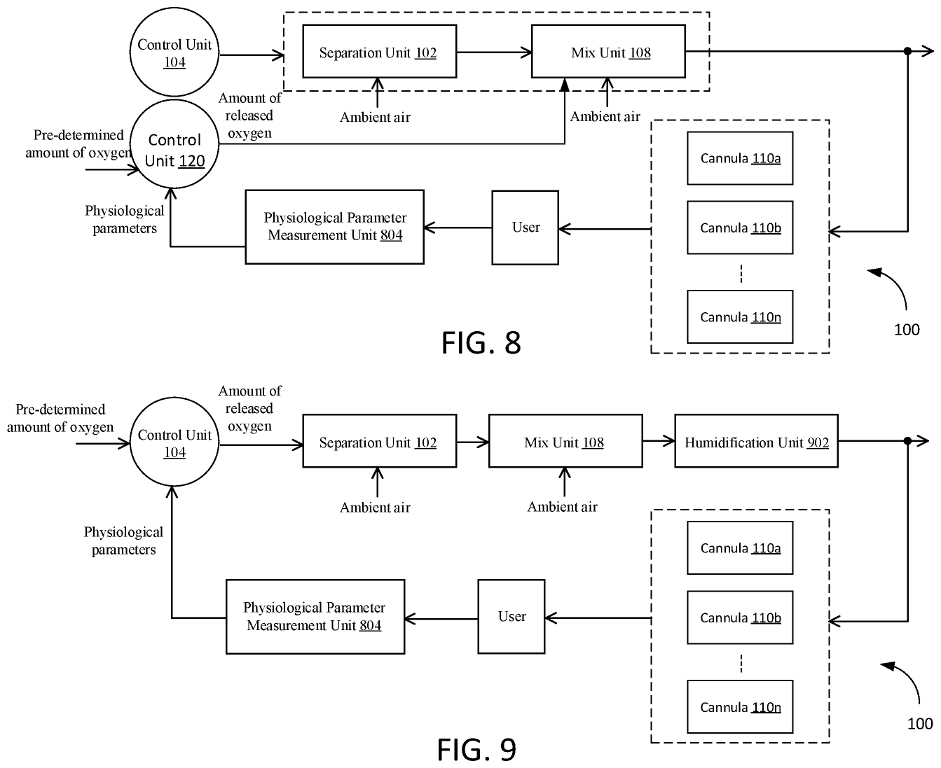

In some embodiments, the nitrogen release unit 106 are controlled by the control unit 104 as to the amount of nitrogen to be released to an environment of the oxygen production unit 100 is located in. Details of some of these embodiments are provided in FIGS. 8-11. In some embodiments, the control unit 104 controls the amount of oxygen and nitrogen separated by the separation unit 102 and/or the mix unit 108. Details of some of these embodiments are provided in FIGS. 8-11. For example, the control unit 104, in one embodiment, controls the amount of amount air flows into the separation unit 102 per second to control the amount of oxygen and nitrogen separated by the separation unit 102. In some embodiments, the control unit 104 control a ratio of oxygen mixed by the mix unit 108. For example, the control unit 104, in one embodiment, controls the amount of amount air flows into the mix unit 108 per second to control the ration of the oxygen in the mixed air.

The cannulas 110a-n may comprise one or more inlets connectable to one or more nitrogen release unit outlets to form one or more nitrogen release unit to cannula channels, and one or more inlets connectable to one or more mix unit outlets to form one or more mix unit to cannula channels, and/or any other inlets. The one or more nitrogen release unit to cannula channels may be configured to facilitate the released nitrogen from the nitrogen release unit 106 into the cannulas 110a-n, and the one or more mix unit to cannula channels may be configured to facilitate the released oxygen from the mix unit 106 into the cannulas 110a-n. An individual cannula, such as the cannula 110a, can be configured to release separated oxygen and separated nitrogen through one or more cannula outlets.

### Cannula Structure

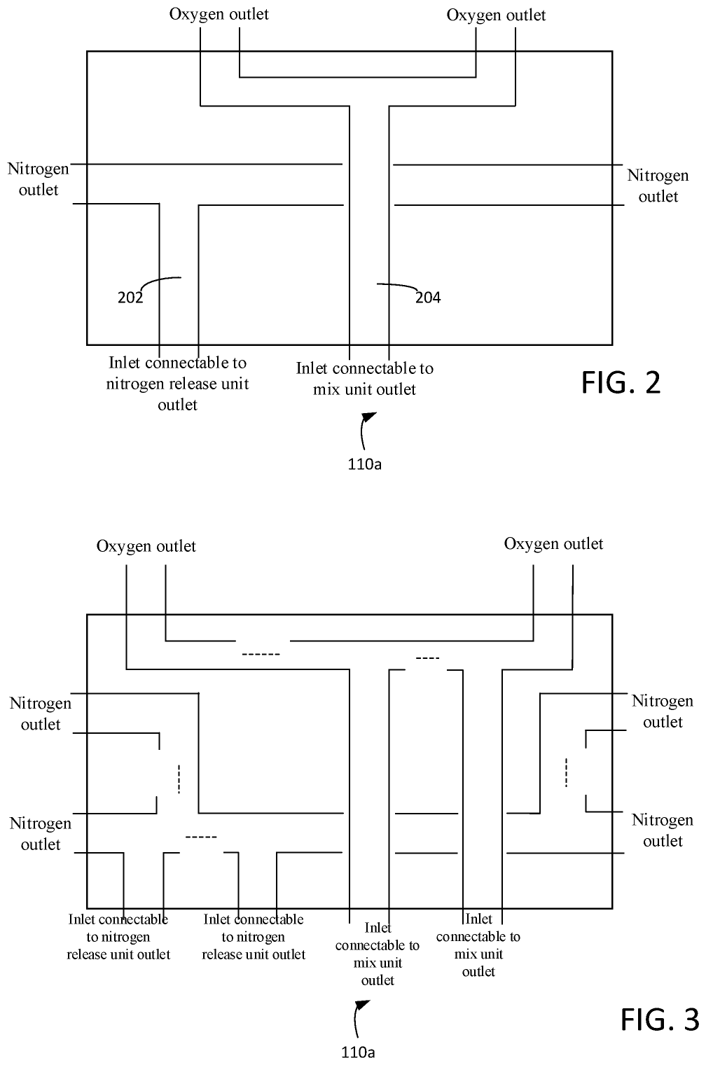

FIG. 2 illustrates an example for the cannula 110a shown in FIG. 1B. As shown, the example cannula 110a shown in FIG. 2 may include one inlet connectable to a nitrogen release unit outlet to form a nitrogen release unit, such as the nitrogen release unit 106 shown in FIG. 1, to cannula channel, one inlet connectable to a mix unit outlet to form a mix unit, such as the mix unit 108 shown in FIG. 1, to cannula channel, two oxygen outlets connectable to two nostrils of the user of the oxygen production unit 100, two nitrogen outlets configured to release the separated nitrogen, and/or any other components.

In this example, the nitrogen release unit to cannula channel 202 and the mix unit to cannula channel 204 are insulated from each other to isolate the separated nitrogen and the separated oxygen. The nitrogen release unit to cannula channel 202 may be configured to facilitate releasing the separated nitrogen within the oxygen production unit 100 and/or into the ambient air through the cannula 110a. When the nitrogen release unit to cannula channel 202 is configured to release the separated nitrogen into the ambient air, high oxygen density in the ambient air can be reduced to enhance safety and prevent fire incidents. When the nitrogen release unit to cannula channel 202 is configured to release the separated nitrogen within the oxygen production unit 100, an inertia gas environment can be created inside the oxygen production unit 100, which can help improve the operation safety of the oxygen production unit 100. The mix unit to cannula channel 204 may be configured to facilitate the separated oxygen into nostrils of the user of the oxygen production unit 100.

The nitrogen outlets shown in this example can be configured and positioned to release the nitrogen from the channel 202 within oxygen production unit 100 and/or into the ambient air. For example, these outlets can be positioned to release the nitrogen around a user or a patient. However, this is not necessarily the only case, as another example, these outlets can be positioned to release the nitrogen within the oxygen production unit 100 and/or into an environment around the oxygen production unit 100 shown in FIG. 1. Other configurations of these outlets are contemplated. It should also be understood that although two nitrogen outlets are shown in FIG. 2, the number of nitrogen outlets in a cannula employed by the oxygen production unit in accordance with the present disclosure is not limited to only two. In various examples, the number of nitrogen outlets in cannula employed by the oxygen production unit in accordance with the present disclosure may be more or less than two.

FIG. 3 illustrates another embodiment for the cannula 110a shown in FIG. 1B. As shown, the example cannula 110a shown in FIG. 3 may include one or more inlets connectable to one or more nitrogen release unit outlets to form one or more nitrogen release unit to cannula channels, one or more inlets connectable to one or more mix unit outlets to form one or more mix unit to cannula channels, one or more oxygen outlets connectable to one or more nostrils of one or more users of the oxygen production unit 100, one or more nitrogen outlets configured to release nitrogen, and/or any other components.

In this embodiment, the one or more nitrogen release unit to cannula channels and the one or more mix unit to cannula channels are insulated to isolate the separated nitrogen and the separated oxygen. The one or more nitrogen release unit to cannula channels may be configured to facilitate releasing the separated nitrogen within the oxygen production unit 100 and/or into the ambient air of the oxygen production unit 100. The one or more mix unit to cannula channels may be configured to facilitate the separated oxygen into the nostrils of one or more users of the oxygen production unit 100.

### Examples of Nitrogen Release Unit

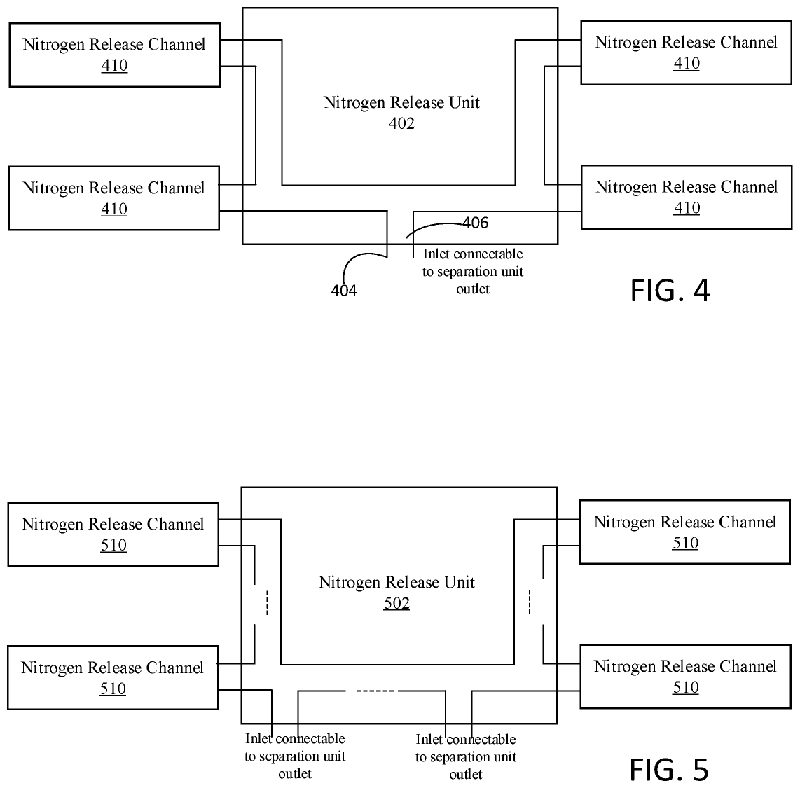

FIG. 4 illustrates an example for the nitrogen release unit 106 shown in FIG. 1B. As shown, the nitrogen release unit 402 shown in FIG. 4 includes four nitrogen release channels 410 at four corners of the nitrogen release unit 106, and/or any other components. A nitrogen release channel 410 may be referred to an outlet in the nitrogen release unit 106. Example of the nitrogen release channel 410 may include a pipe outlet, and/or any other types of outlets. In various embodiments, the nitrogen release channel 410 may be configured to facilitate direct release of the separated nitrogen from the nitrogen release unit 106 into an ambient environment of the oxygen production unit 100. Direct release of the separated nitrogen from the nitrogen release unit 106 into the ambient environment of the oxygen production unit 100 may be referred to release of the separated nitrogen from the nitrogen release unit 106 directly to the ambient environment of the oxygen production unit 100 without passing the separated nitrogen through any other tubes or cannulas. However, this is not necessarily the only case. In some embodiments, the nitrogen release channel 410 may be arranged within the oxygen production unit 100 to facilitate circulation of nitrogen within the oxygen production unit 100. In those embodiments, an inertia gas environment can be created inside within the oxygen production unit 100 through the circulation of the nitrogen. This can help improve operation safety of the oxygen production unit 100.

As shown, in various embodiments, the nitrogen release unit 402 includes an inlet 404 in order to receive and/or obtain the separated nitrogen from an outlet of a separation unit, such as the separation unit 102 shown in FIG. 1, through the separation unit to nitrogen release unit channel, such as channel 406 shown. Example of the separation unit to nitrogen release unit channel 406 may include a pipe configured to connect the outlet of the separation unit 102 and the inlet of the nitrogen release unit 106. The pipe may be configured to allow the separated nitrogen to pass from the separation unit 102 to the nitrogen release unit 106. In this example, the nitrogen release unit inlet 402 may be configured to be connected to the nitrogen release channels 410 to facilitate direct release of the separated nitrogen from the separation unit 102. As mentioned above, in some embodiments, the release of the separated nitrogen from the separation unit 102 by the nitrogen release channels 410 may include releasing the separated nitrogen into the ambient environment of the oxygen production unit 100; and in some embodiments, he release of the separated nitrogen from the separation unit 102 by the nitrogen release channels 410 may include releasing nitrogen within the oxygen production unit 100, which may or may not include releasing the nitrogen into the ambient environment.

FIG. 5 illustrates another embodiment for the nitrogen release unit 106 shown in FIG. 1. As shown, the nitrogen release unit 502 shown in FIG. 5 includes one or more nitrogen release channels 410, one or more inlets configured to receive and/or obtain the separated nitrogen from one or more outlets in a separation unit, such as the separation unit 102 shown in FIG. 1, through one or more separation unit to nitrogen release unit channels, and/or any other components. Examples of separation unit to nitrogen release unit channels may include pipes configured to connect the separation unit 102 and the nitrogen release unit 106. The pipes may be configured to allow the separated nitrogen to pass from the separation unit 102 to the nitrogen release unit 106.

In this embodiment, the nitrogen release channels 510 may be configured facilitate release of the separated nitrogen, for example, into the ambient environment of the oxygen production unit 100 and/or, for example, within the oxygen production unit 100. The one or more inlets in the nitrogen release unit 106 may be configured to be connected to the one or more nitrogen release channels 510. In this embodiment, the one or more nitrogen release channels 510 may be configured to facilitate release of the separated nitrogen through the separation unit to nitrogen release unit channels and the nitrogen release channels 510 without passing the separated nitrogen through any other tubes or cannulas. In embodiments where the separated nitrogen is released to the ambient environment, the nitrogen released by the nitrogen release unit 502 may help create a shield around the oxygen production unit 100 to protect the oxygen production unit 100 from potential fire hazards by reducing the oxygen density around the oxygen production unit 100. In embodiments where the separated nitrogen is released within the oxygen production unit 100, an inertia gas environment may be created inside the oxygen production unit 100, which can help improve operation safety of the oxygen production unit 100.

Nitrogen Release Mechanism with Adsorbent Unit

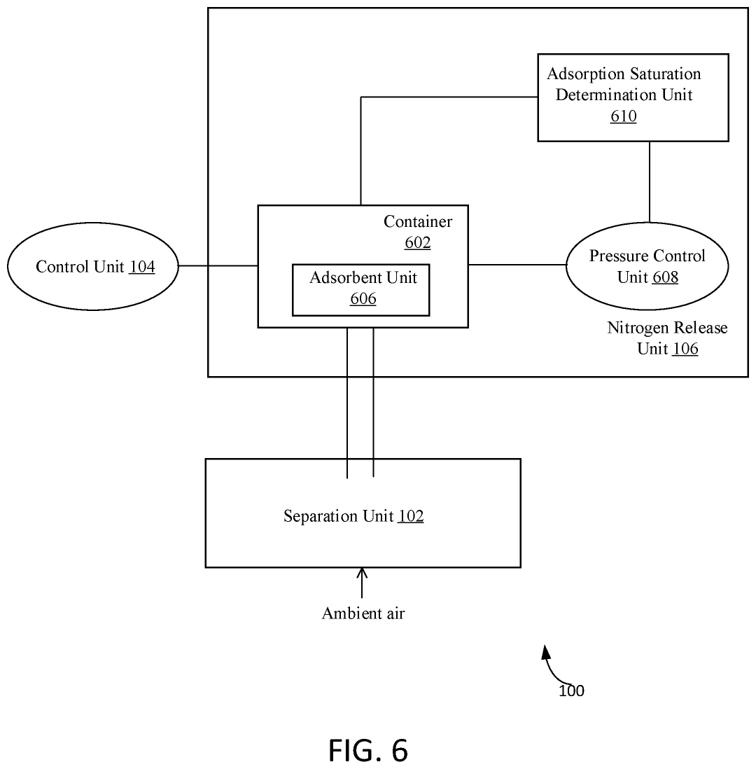

FIG. 6 illustrates an embodiment of the oxygen production unit 100 shown in FIG. 1B. As shown, in this embodiment, the oxygen production unit 100 includes a separation unit 102, a control unit 104, a nitrogen release unit 106, and/or any another components. In this example, the nitrogen release unit 106 includes an adsorbent unit 606, a pressure control unit 608, an adsorption saturation determination unit 610, and/or any other components. In various embodiments, the container 602 may include a pressure vessel for storage and containment of gas at a predetermined pressure level. In this embodiment, the container 602 is configured to receive and/or obtain container control signals from the control unit 104. Container control signals may include container release start and stop signal, air pressure generation signal, air pressure adjustment control signal, container release runtime control signal, emergent stop signal, and/or any other container control signals.

In this embodiment, the container 602 includes an adsorbent unit 606. An adsorbent unit 606 may include adsorbent materials configured to adsorb target gas at high pressure and desorb the adsorbed gas at low pressure. Examples of the adsorbent unit 606 may include Metal-Organic Frameworks (MOFs) or Covalent Organic Frameworks (COFs) configured to adsorb nitrogen and pass oxygen, resulting in simultaneous separation of nitrogen from oxygen, and/or any other types of adsorbent materials. The pressure control unit 608 may be referred to an air compressor configured to adjust air pressure in the container 602.

The adsorption saturation determination unit 610 may include an instrument configured to determine whether saturation occurs in the adsorbent unit 606. Example of the adsorption saturation determination unit 610 may include an instrument configured to calculate time to saturation based on the adsorbent materials in the adsorbent unit 606, temperature in the container 602, air pressure in the container 602, and/or any other parameters. In this example, the pressure control unit 608 may be configured to receive and/or obtain a saturation signal from the adsorption saturation determination unit 610 when saturation in the adsorbent unit 606 is detected by the adsorption saturation determination unit 610. Saturation in the adsorbent unit 606 may be referred to a state of the adsorbent unit 606 when molecules of the nitrogen reach the maximum amount on the surface of the adsorbent materials in the adsorbent unit 606 for a given air pressure and temperature value.

The adsorption saturation determination unit 610 may be configured to be operatively connected to the container 602 in order to determine whether saturation in the adsorbent unit 606 occurs. The adsorption saturation determination unit 610 may be configured to be operatively connected to the pressure control unit 608 in order to send a saturation status signal to the pressure control unit 608. A saturation status signal may be referred to a binary signal such that a zero in the saturation status signal indicates non-saturation, and a one in the saturation status signal indicates saturation. When the saturation signal is zero as determined by the adsorption saturation determination unit 610, the pressure control unit 608 may be configured to increase air pressure in the container 602 or maintain air pressure in the container 602 so that the adsorbent unit 606 may continue to adsorb nitrogen. When the saturation signal is one as determined by the adsorption saturation determination unit 610, the pressure control unit 608 may be configured to decrease air pressure in the container 602 in order to release the separated nitrogen from the adsorbent unit 606 to the container 602.

In this embodiment, the separation unit 102 comprises a third separation unit outlet connectable to a first container inlet of the container 602 to form a first separation unit to container channel. The first separation unit to container channel may be configured to facilitate ambient air from the separation unit 102 into the container 602. The pressure control unit 608 may comprise a first pressure control unit outlet connectable to a second container inlet of the container 602 to form a first pressure control unit to container channel. The first pressure control unit to container channel may be configured to facilitate compressed air from the pressure control unit 608 to the container 602 in order to adjust air pressure in the container 602.

Nitrogen Release Mechanism with Outlet

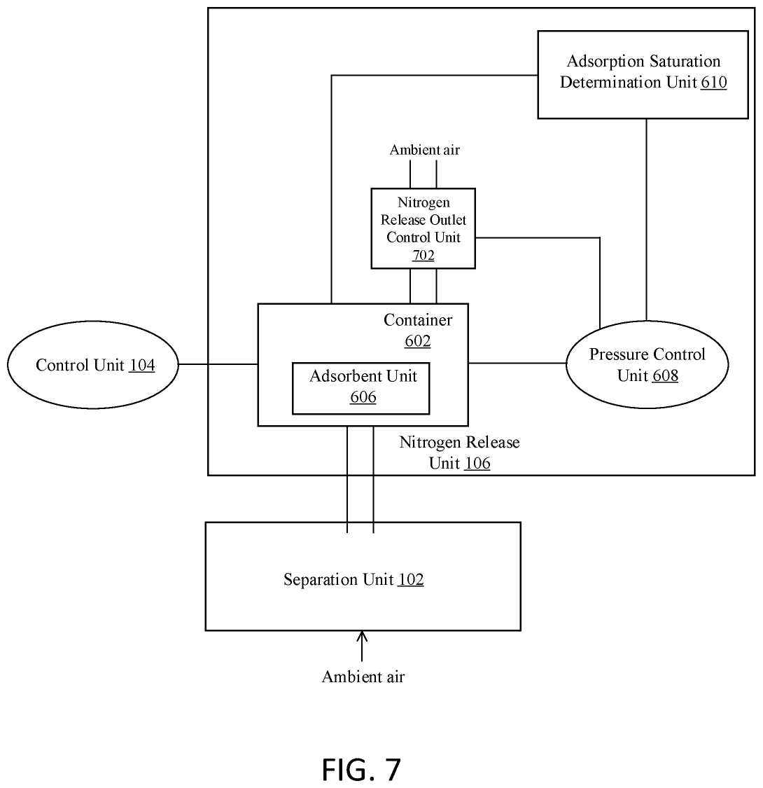

FIG. 7 illustrates an embodiment of the oxygen production unit 100 shown in FIG. 1B. As shown, in this example, the oxygen production unit 100 includes a control unit 104, a nitrogen release unit 106, and/or any other components. In this example, the nitrogen release unit 106 includes a container 602, a pressure control unit 608, an adsorption saturation determination unit 610, a nitrogen release outlet control unit 702, and/or any other components. In this embodiment, the nitrogen release outlet control unit 702 is configured to be connectable to a first container outlet of the container 602 to form a first container to nitrogen release outlet channel. The first container to nitrogen release outlet channel may be configured to facilitate releasing the separated nitrogen from the container 602 within the oxygen production unit 100 and/or into the ambient air through the nitrogen release outlet control unit 702. The nitrogen control release unit 702 may be configured to control an amount of nitrogen released to an environment of the oxygen production unit during a unit period of time. For example, the nitrogen control release unit 702 may be configured to control the amount of nitrogen to be released according to a predetermined setting set by an operator of the oxygen production unit 100 (such as the user of oxygen production unit 100 or any other operator). As another example, the oxygen production unit 100 may be configured to automatically adjust the amount of the nitrogen released to the environment based on the oxygen density in the environment.

### Feedback Loop Control

FIG. 8 illustrates an example implementation for the oxygen production unit 100 shown in FIG. 1C. As shown, in this example may include a control unit 104, a separation unit 102, a mix unit 108, a control unit 120 one or more cannulas such as 110a, 110b and 110n shown, a physiological parameter measurement unit 804, and/or any other components. The control unit 120 may be configured to receive and/or obtain a value indicating a pre-determined amount of oxygen to be released to the user of the oxygen production unit 100, one or more physiological parameter values from the physiological parameter measurement unit 804, and/or any other parameter values. As will be described below, the control unit 120 is this embodiment is configured to control an amount of oxygen to be delivered to the user by way of controlling the separation unit 102, and the mix unit 108.

The physiological parameter measurement unit 804 may include one or more measurement instruments configured to measure one or more physiological parameters of the user of the oxygen production unit 100. The physiological parameter measurement unit 804 may include pulse oximeter, spirometer, heart rate monitor, and/or any other physiological parameter measurement instruments. The one or more physiological parameters may be referred to parameters indicating physical health conditions of the user. Examples of physiological parameters may include blood oxygen saturation, respiratory rate, heart rate, blood pressure, and/or any other physiological parameters.

In this example, the control unit 104 is operatively connected to the separation unit 102 and the mix unit 108. The separation unit 102 in this embodiment is configured to receive and/or obtain an amount of released oxygen from the control unit 104. The mix unit 108 in this embodiment is configured to receive and/or obtain an amount of released oxygen from the control unit 104. The amount of released oxygen may be referred to a parameter indicating amount of oxygen to be released to the user. The separation unit 102 may comprise a second separation unit outlet connectable to an inlet of the mix unit 108 to form a separation unit to mix unit channel. The separation unit to mix unit channel may be configured to facilitate the separated oxygen from the separation unit 102 to the mix unit 108. The separation unit 102 may be configured to receive and/or obtain ambient air to produce separated oxygen and separated nitrogen. The mix unit 108 may be configured to receive and/or obtain ambient air in order to adjust the amount of the separated oxygen based on the amount of released oxygen received and/or obtained from the control unit 104.

The mix unit 108 may comprise one or more outlets connectable to one or more inlets of the cannulas 110a-n to form one or more mix unit to cannula channels. In this embodiment, the one or more mix unit to cannula channels may be configured to facilitate the separated oxygen from the mix unit 108 into the cannulas 110a-n. The cannulas 110a-n may be configured to be connected to one or more nostrils of the user of the oxygen production unit 100, and the physiological parameter measurement unit 804 may be configured to measure one or more physiological parameters of the user. Examples of physiological parameter may include blood oxygen saturation, respiratory rate, heart rate, blood pressure, and/or any other physiological parameters.

In this example, the control unit 110 is configured to receive and/or obtain one or more physiological parameter values from the physiological parameter measurement unit 804. Based on the one or more physiological parameter values received and/or obtained from the physiological parameter measurement unit 804 and the value indicating the pre-determined amount of oxygen to be released, the control unit 104 may be configured to adjust the amount of oxygen to be released to the user of the oxygen production unit 100.

FIG. 9 illustrates another embodiment of the oxygen production unit 100 shown in FIG. 1B. As shown, in this embodiment, the oxygen production unit 100 includes a control unit 104, a separation unit 102, a mix unit 108, a humidification unit 902, one or more cannulas such as 110a, b and n shown, a user of the oxygen production unit 100, a physiological parameter measurement unit 804, and/or any other components. In this embodiment, the control unit 104 is configured to receive and/or obtain a value indicating a pre-determined amount of oxygen to be released to the user of the oxygen production unit 100, one or more physiological parameter values from the physiological parameter measurement unit 904, and/or any other parameter values.

In this embodiment, the humidification unit 902 comprises one or more outlets connectable to one or more inlets of the cannulas 110a-n to form one or more humidification unit to cannula channels. The one or more humidification unit to cannula channels may be configured to facilitate the separated and humidified oxygen from the humidification unit 802 into the cannulas 110a-n. Please reference FIG. 8 and its associated texts for structure and functions of other components included in this embodiment.

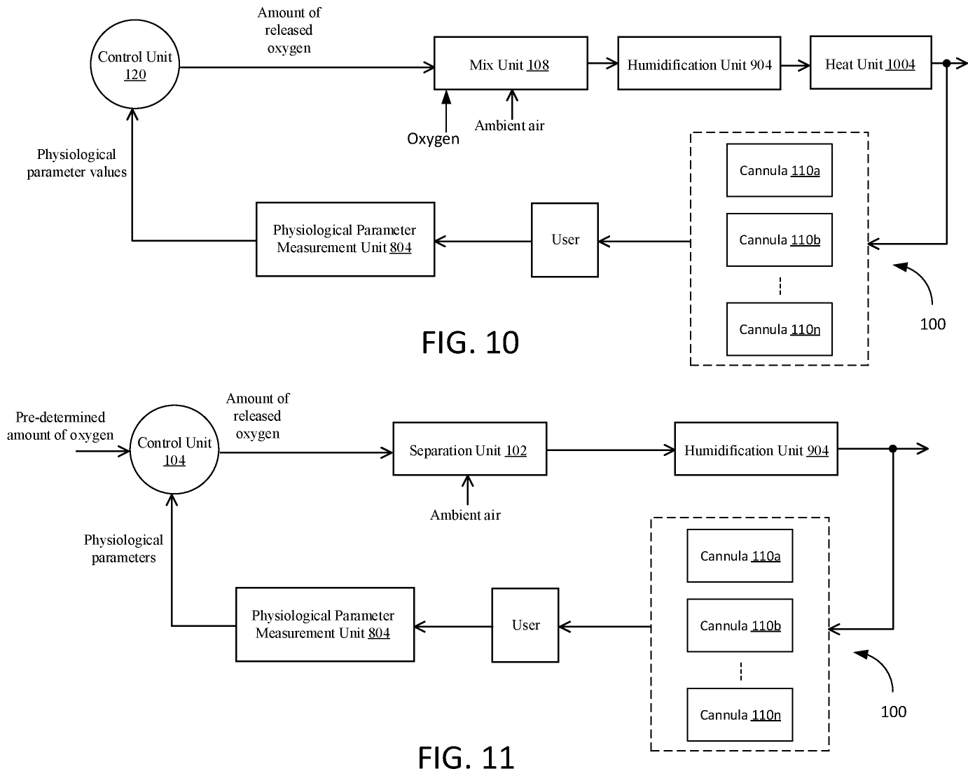

FIG. 10 illustrates an embodiment for the oxygen production unit 100 shown in FIG. 1C. As shown, in this embodiment, the oxygen production unit 100 includes a control unit 110, a mix unit 108, a humidification unit 904, a heat unit 1004, one or more cannulas such as 110a, b and n shown, a user of the oxygen production unit 100, a physiological parameter measurement unit 804, and/or any other components.

In this embodiment, the control unit 120 is configured to control mixing of the ambient air and oxygen (from the separation unit 102 for example) to achieve a desired ratio of oxygen to be released to the patient. The heat unit 1004 comprises one or more outlets connectable to one or more inlets of the cannulas 110a-n to form one or more heat unit to cannula channels. The one or more heat unit to cannula channels may be configured to facilitate the separated, humidified, and heated oxygen from the heat unit 1002 into the cannulas 110a-n. Please reference FIG. 8 and FIG. 9 and their associated texts for structure and functions of other components included in this embodiment.

FIG. 11 illustrates another embodiment of the oxygen production unit 100 shown in FIG. 1. As shown, in this embodiment, the oxygen production unit 100 includes a control unit 104, a separation unit 102, a humidification unit 904, one or more cannulas such as 110a, b and n shown, a physiological parameter measurement unit 804, and/or any other components. In this embodiment, the control unit 104 is configured to receive and/or obtain a value indicating a predetermined amount of oxygen to be released to the user of the oxygen production unit 100, one or more physiological parameter values from the physiological parameter measurement unit 804, and/or any other parameter values.

In this embodiment, the control unit 104 is operatively connected to the separation unit 102. The separation unit 102 is configured to receive and/or obtain an amount of released oxygen from the control unit 104. In this embodiment, the humidification unit 1102 comprises one or more outlets connectable to one or more inlets of the cannulas 110a-n to form one or more humidification unit to cannula channels. The one or more humidification unit to cannula channels may be configured to facilitate the separated and humidified oxygen from the humidification unit 802 into the cannulas 110a-n.

The cannulas 110a-n may be configured to be connected to one or more nostrils of the user of the oxygen production unit 100, and the physiological parameter measurement unit 804 may be configured to measure one or more physiological parameters of the user. Examples of physiological parameter may include blood oxygen saturation, respiratory rate, heart rate, blood pressure, and/or any other physiological parameters.

In this example, the control unit 104 is configured to receive and/or obtain one or more physiological parameter values from the physiological parameter measurement unit 804. Based on the one or more physiological parameter values received and/or obtained from the physiological parameter measurement unit 804 and the value indicating the pre-determined amount of oxygen to be released, the control unit 104 may be configured to adjust the amount of oxygen to be released to the user of the oxygen production unit 100.

### Automatic Determination of Oxygen Amount

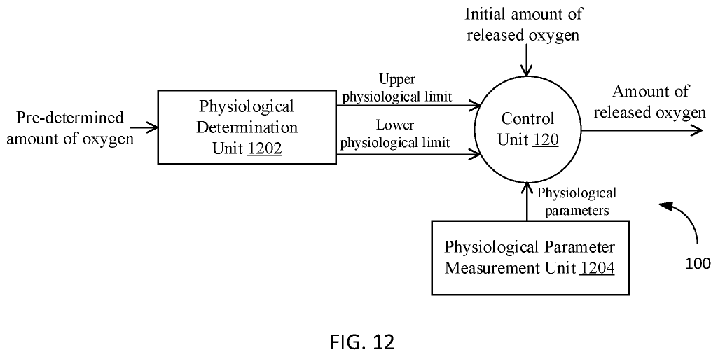

FIG. 12 illustrates an embodiment of the oxygen production unit 100 shown in FIG. 1B. As shown, in this embodiment, the oxygen production 100 unit includes a control unit 104, a physiological determination unit 1202, a physiological parameter measurement unit 1204, and/or any other components. The physiological determination unit 1202 may include a logic configured to determine an upper physiological limit and a lower physiological limit based on a pre-determined amount of oxygen. For example, the physiological determination unit 1202 may include a micro-processor configured to execute machine-readable instructions.

Example of the lower physiological limit may include a lower limit for blood oxygen saturation level denoted by bₗ. Example of the higher physiological limit may include a higher limit for blood oxygen saturation level denoted by bₕ. Example of the pre-determined amount of oxygen may include a scalar value indicated by o. In this embodiment, the lower physiological limit bₗ may be determined by the pre-determined amount of oxygen o based on a first polynomial function bₗ=f_{low}(o)=aₘo^{m}+aₘ₋₁o^{m-1}+ . . . +a₁o+a₀ where aₘ, aₘ₋₁, . . . , a₁, a₀ are constant coefficients of the polynomial function f_{low}, and m is the order of the polynomial function f_{low}. The higher physiological limit bₕ may be determined by the pre-determined amount of oxygen o based on a second polynomial function bₕ=f_{high}(o)=bₘo^{m}+bₘ₋₁o^{m-1}+ . . . +b₁o+b₀ where bₘ, bₘ₋₁, . . . , b₁, b₀ are constant coefficients of the polynomial function f_{high}, and m is the order of the polynomial function f_{high}.

In this embodiment, the control unit 110 may be configured to receive and/or obtain a pre-determined amount of oxygen to be released to the user of the oxygen production unit 100, one or more physiological parameter values from the physiological parameter measurement unit 1204, an initial amount of released oxygen, and/or any other parameter values. The initial amount of released oxygen may be referred to a default value for the amount of oxygen to be released.

The physiological parameter measurement unit 1204 may include one or more measurement instruments configured to measure one or more physiological parameters of the user of the oxygen production unit 100. The physiological parameter measurement unit 1204 may include pulse oximeter, spirometer, heart rate monitor, and/or any other physiological parameter measurement instruments. The one or more physiological parameters may be referred to parameters indicating physical health conditions of the user. Examples of physiological parameters may include blood oxygen saturation, respiratory rate, heart rate, blood pressure, and/or any other physiological parameters.

In this example, the control unit 104 is configured to determine the amount of released oxygen based on the initial amount of released oxygen, the upper physiological limit, the lower physiological limit, one or more physiological parameter values, and/or any other parameter values. The physiological parameter measurement unit 804 may be configured to update the one or more physiological parameter values at a pre-determined period value. Examples of predetermined period values may include every one second, every two seconds, and/or any other period values. Based on the updated one or more physiological parameter values, the control unit 104 may be configured to update/adjust the amount of released oxygen. Algorithm 1 illustrates an example of pseudocode of the control unit 104 to automatically determine the amount of oxygen to be released to the user of the oxygen production unit 100.

Algorithm 1 Example of pseudo code of the control unit 104.

```
INPUT Pre_determ_oxygen, Physio_param
DEFINE Initial_oxygen, oxygen_update_step
Lower_lmt_physio = f_low(Pre_determ_oxygen)
Upper_lmt_physio = f_high(Pre_determ_oxygen)
Release_oxygen = Initial_oxygen
REPEAT
  WHILE (Physio_param >= Lower_lmt_physio AND
  Physio_param <=
  Upper_lmt_physio) DO nothing
  WHILE Physio_param > Upper_lmt_physio DO
    Release_oxygen = Release_oxygen − oxygen_update_step
  WHILE Physio_param < Lower_lmt_physio DO
    Release_oxygen = Release_oxygen + oxygen_update_step
OUTPUT Release_oxygen
```

### Control Unit for Nitrogen Saturation Determination

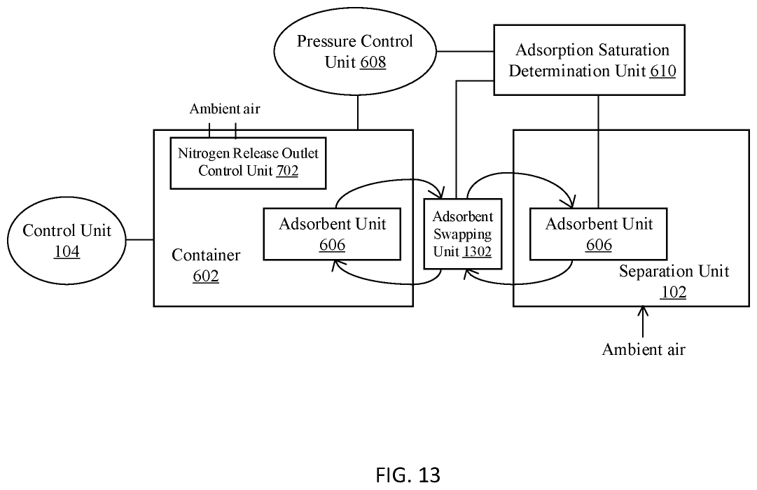

FIG. 13 illustrates an embodiment of the oxygen production unit 100 shown in FIG. 1A. As shown, the embodiment of the oxygen production unit 100 includes a container 602 configured to include a first adsorbent unit 606 and a nitrogen release outlet control unit 702, a control unit 104, a pressure control unit 608, a separation unit 102 configured to include a second adsorbent unit 606, an adsorption saturation determination unit 610, an adsorbent swapping unit 1302 and/or any other components.

In this embodiment, the separation unit 102 is configured to receive and/or obtain ambient air through one or more separation unit inlets. The pressure control unit 608 may be configured to be operatively connected to the adsorption saturation determination unit 610 and the container 602. The adsorbent swapping unit 1302 may be configured to be operatively connected to the adsorption saturation determination unit 610. The adsorbent swapping unit 1302 may be referred to a machine configured to swap the first adsorbent unit 606 in the container 602 and the second adsorbent unit 606 in the separation unit 102 based on an adsorbent unit 606 saturation status signal received and/or obtained from the adsorption saturation determination unit 610. An adsorbent unit 606 saturation status signal may be referred to a binary signal such that a zero value indicates non-saturation in the adsorbent unit 606, and a one value indicates saturation in the adsorbent unit 606.

The adsorption saturation determination unit 610 may be configured to be operatively connected to the separation unit 102 in order to determine whether saturation in the adsorbent unit 606 in the separation unit 102 occurs. Example of the adsorption saturation determination unit 610 may include an instrument configured to calculate time to saturation based on the adsorbent materials in the adsorbent unit 606, temperature in the separation unit 102, air pressure in the separation unit 102, and/or any other parameters. Saturation in the adsorbent unit 606 may be referred to a state of the adsorbent unit 606 when molecules of the nitrogen reach the maximum amount on the surface of the adsorbent materials in the adsorbent unit 606 for a given air pressure and temperature value. An adsorbent unit 606 may be referred to adsorbent materials configured to adsorb target gas at high pressure and desorb the adsorbed gas at low pressure. Examples of the adsorbent unit 606 may include Metal-Organic Frameworks (MOFs) or Covalent Organic Frameworks (COFs) configured to adsorb nitrogen and pass oxygen, resulting in simultaneous separation of nitrogen from oxygen, and/or any other types of adsorbent materials.

When the adsorbent unit 606 saturation status signal value is one as determined by the adsorption saturation determination unit 610, the adsorbent swapping unit 1302 may be then configured to swap the first adsorbent unit 606 in the container 602 and the second adsorbent unit 606 in the separation unit 102. When the first adsorbent unit 606 in the container 602 and the second adsorbent unit 606 in the separation unit 102 are swapped by the adsorbent swapping unit 1302, the pressure control unit 608 may be configured to decrease air pressure in the container 602 in order to release the separated nitrogen from the swapped second adsorbent unit 606 to the container 602. The released nitrogen in the container 602 may be then released from the container 602 within the oxygen production unit 100 and/or into the ambient environment through the nitrogen release outlet control unit 702.

In this embodiment, the container 602 may be configured to receive and/or obtain container control signals from the control unit 104. Container control signals may include container release start and stop signal, air pressure generation signal, air pressure adjustment signal, container release runtime control signal, emergent stop signal, and/or any other container control signals. The nitrogen release outlet control unit 702 may be configured to be connectable to a first container outlet of the container 602 to form a first container to nitrogen release outlet channel. The first container to nitrogen release outlet channel may be configured to facilitate releasing the separated nitrogen from the container 602 within the oxygen production unit 100 and/or into the ambient environment.

### Mix Unit Control

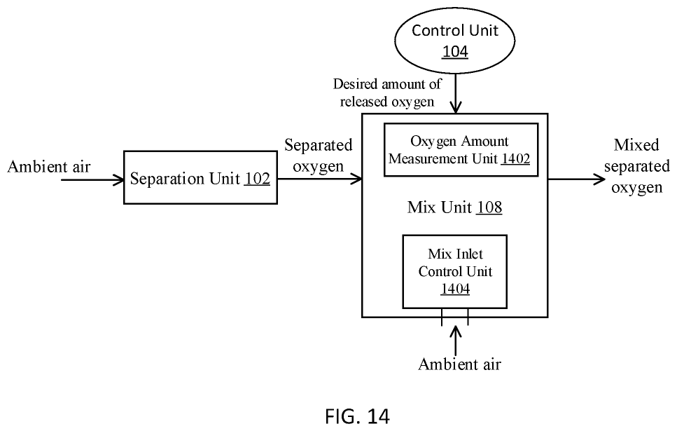

FIG. 14 illustrates an embodiment of oxygen production unit 100 shown in FIG. 1. As shown, in this embodiment, the oxygen production unit 100 includes a control unit 104, a separation unit 102, a mix unit 108 configured to include an oxygen amount measurement unit 1402 and a mix inlet control unit 1404, and/or any other components. The oxygen amount measurement unit 1402 may include an instrument configured to measure amount of oxygen in the mix unit 108. An amount of oxygen in the mix unit 108 may be referred to a ratio between volume of oxygen and volume of all air constituents in the mix unit 108. Examples of the oxygen amount measurement unit 1402 may include electro-galvanic oxygen sensor, electrochemical oxygen sensor, and/or any other types of instruments.

The mix inlet control unit 1404 may include an inlet in the mix unit 108 configured to receive an inlet control signal from the mix unit 108 to control passage of ambient air from ambient environment into the mix unit 108. An inlet control signal may be referred to a binary signal such that a zero in the inlet control signal value closes the inlet in mix inlet control unit 1404 to prevent ambient air from entering the mix unit 108, and a one in the inlet control signal value opens the inlet in the mix inlet control unit 1404 to facilitate ambient air from ambient environment into the mix unit 108.

The separation unit 102 may comprise one or more inlets to receive and/or obtain ambient air, and a second separation unit outlet connectable to a first mix unit inlet of the mix unit 108 to form a first separation unit to mix unit channel. The first separation unit to mix unit channel may be configured to facilitate the separated oxygen from the separation unit 102 to the mix unit 108. The mix unit 108 may be configured to receive and/or obtain a value of desired amount of released oxygen from the control unit 104.

In this example, the mix unit 108 may be configured to receive and/or obtain measured amount of oxygen in the mix unit 108 from the oxygen amount measurement unit 1402 at a predetermined period value. Examples of pre-determined period values may include every one second, every two seconds, and/or any other period values. Upon receiving and/or obtaining the measured amount of oxygen from the oxygen amount measurement unit 1402, the mix unit 108 may be configured to compare the measured amount of oxygen to the desired amount of released oxygen received and/or obtained from the control unit 104. Based on the comparison between the measured amount of oxygen and the desired amount of released oxygen, the mix unit 108 may be configured to update the inlet control signal in the mix inlet control unit 1404. Algorithm 2 illustrates an example of pseudocode in the mix unit 108 in order to control the mix inlet control unit 1404 based on the comparison between the measured amount of oxygen and the desired amount of released oxygen.

Algorithm 2 Example of pseudo code of
the mix unit 108 for mix inlet control.

```
INPUT Desired_oxygen, Measured_oxygen
inlet_ctrl_signal = 0
REPEAT
  WHILE Measured_oxygen <= Desired_oxygen DO nothing
  WHILE Measured_oxygen > Desired_oxygen DO
    inlet_ctrl_signal = 1
OUTPUT inlet_ctrl_signal
```

### Example Method

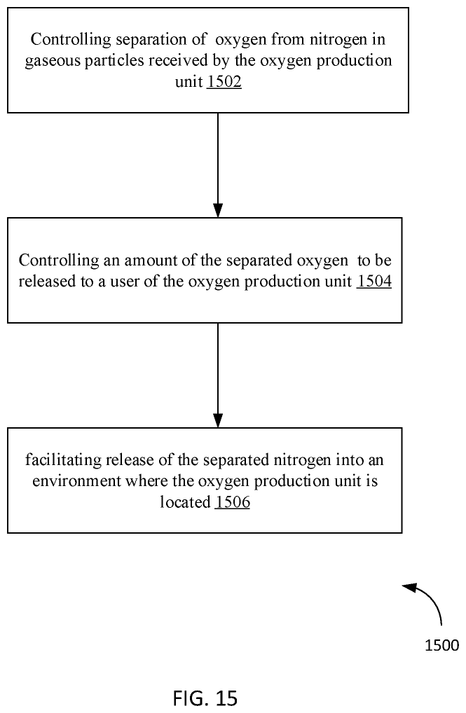

FIG. 15 illustrates one example method for producing oxygen for a user in accordance with the disclosure. Operations shown in FIG. 15 may be implemented, for example, by a control unit the same as or substantially similar to the control unit 104 illustrated and described herein.

At 1502, separation of oxygen from nitrogen in gaseous particles received by the oxygen production is controlled. Details of operations 802 in some embodiments are described in association with FIGS. 8-12.

At 1504, an amount of the separated oxygen to be released to a user of the oxygen production unit is released. Details of operations 802 in some embodiments are described in association with FIGS. 8-12.

At 1506, release of the separated nitrogen into an environment where the oxygen production unit is located is facilitated. Details of operations 802 in some embodiments are described in association with FIGS. 13-14.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

What is claimed is:

1. An oxygen production unit comprising:
a separation unit configured to separate oxygen from nitrogen in received gaseous particles;
a control unit comprising a processor configured to control an amount of the separated oxygen to be released to a user of the oxygen production unit; and
a nitrogen release unit configured to facilitate release of the separated nitrogen into an environment where the oxygen production unit is located.

2. The oxygen production unit of claim 1, wherein the nitrogen release unit comprises a first outlet connectable to a first inlet of a cannula to form a first channel, and a second outlet connectable to a second inlet of the cannula to form a second channel, wherein the first channel facilitates releasing the separated nitrogen within oxygen production unit and/or into the ambient environment through the cannula, and the second channel facilitates the separated oxygen into a nose of the user through the cannula, the first channel and the second channel are insulated from each other.

3. The oxygen production unit of claim 1, wherein the nitrogen release unit comprises an outlet openable to within the oxygen production unit and/or to the ambient environment, and is configured to facilitate the release of the separated nitrogen within the oxygen production unit and/or into the environment.

4. The oxygen production unit of claim 1, wherein the nitrogen release unit comprises a container configured house a piece absorbed with the separated nitrogen; and, wherein facilitating the release of the nitrogen comprises:
generating a pressure difference in the container to facilitate the separated nitrogen to be released into the environment.

5. The oxygen production unit of claim 4, wherein the nitrogen release unit comprises an outlet openable to the ambient environment; and, wherein the separated nitrogen is released from the container to the ambient environment through the outlet.

6. The oxygen production unit of claim 1, wherein the control unit is configured to:
receive a value indicating pre-determined amount of oxygen to be released to the user;
receive a value indicating a physiological parameter regarding the user; and
determine the amount of the oxygen to be released to the user based on the predetermined amount of oxygen and the physiological parameter.

7. The oxygen production unit of claim 6, wherein determining the amount of the oxygen to be released to the user based on the pre-determined amount of oxygen and the physiological parameter comprises:
determining an adjustment value in the amount of the oxygen to be released to the user based on the pre-determined amount of oxygen and the physiological parameter to obtain.

8. The oxygen production unit of claim 4, wherein the control unit is further configured to determine a nitrogen level in the piece exceeds a threshold level and generate a signal to alert that nitrogen level in the piece has exceeded the threshold level.

9. The oxygen production unit of claim 1, further comprising a mix unit configured to:
receive a value indicating the amount of the oxygen to be release to the user from the control unit;
obtain an amount of gas based on the value; and
mix the separated oxygen with the amount of gas to achieve the amount of the oxygen to be release to the user.

10. The oxygen production unit of claim 1, further comprising a humidification unit configured to humidify the separated oxygen before being released to the user.

11. The oxygen production unit of claim 1, further comprising a heat unit configured to heat the separated oxygen before being released to the user.

12. The oxygen production unit of claim 1, further comprising a physiological parameter measurement unit including one or more measurement instruments configured to measure one or more physiological parameters of the user; and, wherein the control unit is configured to control the amount of the separated oxygen to be released to the user based on the physiological parameters.

13. The oxygen production unit of claim 12, further comprising a physiological determination unit configured to determine an upper physiological limit and a lower physiological limit based on a pre-determined amount of oxygen; and, wherein the control unit is configured to control the amount of the separated oxygen to be released to the user based on the upper physiological limit and the lower physiological limit.

14. The oxygen production unit of claim 12, wherein the physiological parameters includes blood oxygen saturation, respiratory rate, heart rate, and/or blood pressure.

15. A method for producing oxygen, the method being implemented by a processor in an oxygen production unit, the method comprising:
controlling separation of oxygen from nitrogen in gaseous particles received by the oxygen production unit;
controlling an amount of the separated oxygen to be released to a user of the oxygen production unit; and
facilitating release of the separated nitrogen into an environment where the oxygen production unit is located.

16. The method of claim 15, wherein oxygen production unit comprises a container configured house a piece absorbed with the separated nitrogen; and, wherein facilitating the release of the nitrogen comprises:
generating a pressure difference in the container to facilitate the separated nitrogen to be released into the environment.

17. The method of claim 15, wherein controlling the amount of the separated oxygen to be released to the user of the oxygen production unit comprises:
receiving a value indicating pre-determined amount of oxygen to be released to the user;
receiving a value indicating a physiological parameter regarding the user; and
determining the amount of the oxygen to be released to the user based on the pre-determined amount of oxygen and the physiological parameter.

18. The method of claim 17, determining the amount of the oxygen to be released to the user based on the pre-determined amount of oxygen and the physiological parameter comprises:
determining an adjustment value in the amount of the oxygen to be released to the user based on the pre-determined amount of oxygen and the physiological parameter to obtain.

19. The method of claim 16, wherein facilitating the release of the separated nitrogen into the environment where the oxygen production unit is located comprises:
determining a nitrogen level in the piece exceeds a threshold level and generate a signal to alert that nitrogen level in the piece has exceeded the threshold level.

20. The method of claim 15, further comprising: determining an upper physiological limit and a lower physiological limit based on a pre-determined amount of oxygen; and, wherein the control the amount of the separated oxygen to be released to the user is based on the upper physiological limit and the lower physiological limit.
